(19) United States
(12) Patent Application Publication
(10) Pub. No.: US 2024/0109702 A1
(43) Pub. Date: Apr. 4, 2024

(54) DOSAGE IN FILM PACKAGE FORM, A FILM COMPOSITION AND A PROCESS FOR PREPARATION THEREOF

(71) Applicant: RKS GLOBAL PTE LTD, Singapore (SG)

(72) Inventors: Rohan Kumar GUPTA, Singapore (SG); Rajiv Kumar GUPTA, Singapore (SG)

(21) Appl. No.: 18/477,724

(22) Filed: Sep. 29, 2023

Related U.S. Application Data
(62) Division of application No. 17/052,864, filed on Nov. 4, 2020.

(30) Foreign Application Priority Data
May 14, 2018 (SG) 10201804058R

(51) Int. Cl. B65D 65/46; A23L 5/42; A23L 25/00; A23L 29/00; A23L 29/256; A23L 29/262; A23L 29/269; A61K 8/02; A61K 8/34; A61K 8/73; A61K 8/9783; B29C 41/00

(52) U.S. Cl. CPC B65D 65/463; A23L 5/42; A23L 25/20; A23L 29/035; A23L 29/256; A23L 29/262; A23L 29/274; A61K 8/0233; A61K 8/345; A61K 8/73; A61K 8/731; A61K 8/733; A61K 8/9783; B29C 41/003; A23V 2002/00; B29K 2001/00

(57) ABSTRACT

The present disclosure envisages an edible film composition for delivering predetermined amount of at least one edible composition to the oral cavity, a packaged dosage form packed in an edible film and a process for preparing the edible film composition and the dosage form, where the edible film composition disintegrates quickly upon placement in the oral cavity. Additionally, the edible composition can be at least one selected from the group consisting of mouth fresheners, mukhawas, mint, chocolate, arica nut, coconut, aftermeal snack or digestive aid, confectioner food product, and fennel seeds.

This application is a divisional application of U.S. Ser. No. 17/052,864, filed Nov. 4, 2020, which is U.S. national stage of PCT/IB2018/058478 filed on 30 Oct. 2018, which claims priority to and the benefit of Singapore Application No. 10201804058R filed 14 May 2018, the contents of which are incorporated herein by reference in their entireties.

### FIELD

The present disclosure relates to a film composition. Particularly to a film which is an edible and a process for preparing thereof.

### Definitions

As used in the present disclosure, the following terms are generally intended to have the meaning as set forth below, except to the extent that the context in which they are used indicate otherwise.

Mukhwas: Mukhwas is a colorful Indian after-meal snack or digestive aid widely used as a mouth freshener, normally after meals. It can be made of various seeds and nuts, but often found with fennel seeds, anise seeds, coconut, areca nut and sesame seeds. It can also be in a powdered, granular form or mixture thereof.

BPA: Bisphenol A is an organic synthetic compound with the chemical formula (CH₃)₂C(C₆H₄OH)₂ belonging to the group of diphenylmethane derivatives and bisphenols, with two hydroxyphenyl groups.

### BACKGROUND

Mukhwas is a colorful Indian after-meal snack or digestive aid widely used as a mouth freshener, normally after meals. It can be made of various seeds and nuts, but often found with fennel seeds, anise seeds, coconut, areca nut and sesame seeds. It can also be in a powdered form. Since the mukhwas is generally a mixture of different edible products of various size and forms. Sometimes people are reluctant to use “Mukhwas” as it may leave a residue on palm which may be valid/applicable for only a few varities of mouth freshners. The intended taste of the mukhwas be provided to the consumer is changed because of the consumption pattern. This generally happens because small particles and powder often sticks to the hand or the spoon, which compromises the intended dosage and consistency and also has an impact on the hygiene, because generally such eatables are taken on the go and mostly without washing of the hands which have a plethora of microorganisms and dirt. Such an action also makes the person susceptible to various spreadable diseases due to the compromised hygiene levels.

Edible films satisfy a variety of needs and meet specific product challenges for a large number of food applications. Edible film packaging has been a topic of interest for many years, though few commercial examples exist. Moreover, these edible compositions can make the excessive packaging unnecessary, which is also perceived as a positive consumer benefit. Today food packaging has become more important than ever. From eco-friendly biodegradable packaging to Bisphenol A (BPA)-free packaging, food companies have been finding alternatives and improvements to their packaging design. Food packaging is important for many reasons; it helps advertise the product, it keeps the food protected from bacteria and it keeps the food fresh and contained—preventing spills and spoilage. Most of the food packagings on the market today are petroleum-based plastics, which are not biodegradable and not environmentally conscious. Edible food packaging eliminates the typical waste cycle and doesn't require any recycling. Since most edible packaging can be eaten or composted, it is very biodegradable and won't fill up landfills or recycling centers.

Further, they can provide good barrier to moisture, oxygen and aroma for food also thereby preventing the loss of taste and spoilage & microbial contamination of the food products. Edible films are promising systems to be used as food carrier. Moreover, such edible packaging does not contribute to the environmental pollution since it is consumed.

Therefore, there exists a need to develop a dosage form comprising of an edible composition in fixed (predetermined) amount and wrapped within an edible film, thereby maintaining the fixed dose/quantity and also maintaining the hygiene.

### Objects

Some of the objects of the present disclosure, which at least one embodiment herein satisfies, are as follows:

It is an object of the present disclosure to ameliorate one or more problems of the prior art or to at least provide a useful alternative.

It is an object of the present disclosure to provide a film composition, which is edible.

It is another object of the present disclosure to provide a dosage form wherein an edible composition is wrapped with the edible film.

It is a yet another object of the present invention to provide an improved, stable and ready to use edible composition wrapped within an edible film.

It is still another object of the present disclosure to provide a process for preparing the edible film composition and the dosage form.

Other objects and advantages of the present disclosure will be more apparent from the following description, which is not intended to limit the scope of the present disclosure.

### SUMMARY

The present disclosure envisages an edible film composition for delivering predetermined amount of at least one edible composition to the oral cavity. The present disclosure also envisages a dosage form comprising the edible composition wrapped within the edible film. Also envisaged is a process for preparing the edible film composition and the dosage form.

The edible film composition, of the present disclosure, comprises a water-dispersible film-forming material. The edible film composition disintegrates quickly upon placement in the oral cavity. In an embodiment of the present disclosure, the edible film composition comprises:
at least one cellulose derivative or cellulose based polymer or polysaccharide polymer or starches;
at least one plasticizer; and
water.

Typically, the edible film composition further comprises at least one flavouring agent and at least one food colouring agent.

In an aspect of the present disclosure, also envisaged is a dosage form. Typically, the dosage form comprises at least one edible composition wrapped within at least one layer of an edible film. Typically, the edible composition can be at least one selected from the group consisting of mouth fresheners, mukhwas, mint, chocolate, Arica nut, coconut, after-meal snack or digestive aid, confectionery food product, and fennel seeds. Typically, the edible composition is wrapped within the edible film manually or mechanically. Typically, the edible film has a thickness ranging from 1 to 30 microns.

In another aspect of the present disclosure, envisaged is a process for preparing the edible film composition.

### DETAILED DESCRIPTION

After meal digestive aids or mouth fresheners, like mukhwas, churans, and the likes, are commonly used in households on daily basis. Generally these digestive aids/mouth fresheners are a mixture of different edible components which are of different types, sizes and consistency. A common method of eating is by hand or by using a spoon. These methods often result in improper amount of the mukhwas or churan being taken and also some particles/amount gets stuck on the hand or the spoon. Further there is improper distribution as smaller particles settle down in the container. All these factors result in poor end user experience, as the intended composition if not available/used by the consumer.

Hence, the inventors of the present disclosure felt a need to develop an edible film which contains a predetermined amount of the digestive aid/mouth freshener. Thus delivering the intended dose in a hygienic pattern to elevate the end user experience.

Accordingly, there exists a need to develop a solution for overcoming the above mentioned drawbacks and which is safe and effective.

The inventors of the present disclosure, in one aspect envisage an edible film composition. The edible film composition of the present disclosure is used for delivering a predetermined amount of at least one edible composition to the oral cavity.

The edible film composition, of the present disclosure, comprises a water-dispersible, water soluble film-forming material. The edible film composition disintegrates quickly upon placement in the oral cavity. In an embodiment of the present disclosure, the edible film composition comprises:
at least one cellulose based polymer or polysaccharides polymer or starches;
at least one plasticizer; and
water.

wherein said edible film composition disintegrates quickly upon placement in the oral cavity without leaving substantial residue which needs to be swallowed or ejected from the mouth. Typically, the edible film composition further comprises at least one flavouring agent and at least one food colouring agent.

In an exemplary embodiment of the present disclosure, the edible film composition for delivering predetermined amount of at least one edible composition to the oral cavity comprises:
a) at least one cellulose derivative or cellulose based polymer or polysaccharide polymer, or blend in the range of 5 to 30 weight % based on the weight of the edible film composition;
   wherein said at least one cellulose derivative or cellulose based polymer or polysaccharide polymer or starches is film forming substance;
b) at least one plasticizer in the range of 1 to 2 weight % based on the weight of the edible film composition;
c) at least one flavouring agent in the range of 0.01 to 1 weight % based on the weight of the edible film composition;
d) at least one food colouring agent in the range of 0.001 to 0.5 weight % based on the weight of the edible film composition; and
e) water as quantum satis (q. s.);
   wherein the edible film composition disintegrates quickly upon placement in the oral cavity without leaving substantial residue or which needs to be swallowed or ejected from the mouth.

Typically, a film forming substance is selected from the group consisting of polysaccharide polymer, pullulan, algenates, cellulose based polymers, cellulose derivatives like carboxy methyl cellulose & its metal salt derivatives, Hydroxy propyl cellulose (HPC), and Hydroxy propyl methyl cellulose (HPMC).

Typically, the plasticizer is selected from the group consisting of glycerol, triacetin, metal salts of fatty acids.

Typically, the flavouring agent is selected from the group consisting of natural extracts or flavors, nature identical extracts or flavors or artificial flavours.

Typically, the food colouring agent is synthetic or natural.

In another aspect of the present disclosure envisaged is a dosage form, comprising the edible composition wrapped within the edible film.

In another exemplary embodiment of the present disclosure, the dosage form comprises at least one edible composition wrapped within at least one layer of an edible film.

In yet another exemplary embodiment of the present disclosure, the dosage form comprises:
at least one layer of an edible film composition;
wherein the edible film composition disintegrates quickly upon placement in the oral cavity without leaving substantial residue which needs to be swallowed or ejected from the mouth
wherein the edible film comprises:
   a) at least one cellulose derivative or cellulose based polymer or polysaccharide polymer or its blend in the range of 5 to 30 weight % based on the weight of the edible film composition;
   b) at least one plasticizer in the range of 1 to 2 weight % based on the weight of the edible film composition;
   c) at least one flavouring agent in the range of 0.01 to 1 weight % based on the weight of the edible film composition;
   d) at least one food colouring agent in the range of 0.001 to 0.5 weight % based on the weight of the edible film composition; and
   e) water as quantum satis(q.s.);
and
at least one edible composition.

In another embodiment of the present disclosure, the edible composition can be at least one selected from the group consisting of, but not limited to, mouth fresheners, mukhwas, mint, chocolate, arica nut, coconut, after-meal snack or digestive aid, confectionery food product, and fennel seeds.

In still another embodiment of the present disclosure, the edible composition is wrapped within the edible film. Typically, the edible composition is wrapped within the edible film manually or mechanically. Typically, this is the dosage form.

In yet another embodiment of the present disclosure, the edible film has a thickness ranging from 1 to 30 microns.

In an embodiment of the present disclosure, the edible composition may be sugar coated and then wrapped with the edible film.

In yet another aspect of the present disclosure envisaged is a process for preparing the edible film composition and the dosage form.

Typically, the process comprises the following steps:
adding in a reaction vessel, predetermined amounts of cellulose derivative and water under continuous stirring conditions to obtain a homogenous mixture;
adding predetermined amount of at least one plasticizer, predetermined amount at least one flavouring agent and predetermined amount at least one food colouring agent to said homogenous mixture to obtain a second homogenous mixture;
de-aereating said second homogenous mixture under vacuum
coating said de-aerated mixture on a PET (polyethylene terephthalate) substrate and drying using hot air;
peeling said coated film to obtain said edible film composition.

In one embodiment of the present disclosure, the edible film composition comprising colours and flavours are shredded into small pieces to make edible glitters or flavouring films.

The present disclosure is further described in light of the following laboratory scale experiments which are set forth for illustration purpose only and not to be construed for limiting the scope of the disclosure. The following experiments can be scaled up to industrial/commercial scale and the results obtained can be extrapolated to industrial/commercial scale.

### Experiment 1

In a reaction vessel, 18 grams of Polysaccharide Polymer was slowly mixed with 100 ml of water at 100 to 200 rpm to obtain a homogenous mixture. 2 grams of glycerol, 0.2 grams of flavour extract, and 0.05 grams of food colour colouring agent, was then added to the homogenous mixture and stirred at 100 to 200 rpm to obtain a second homogenous mixture. This was followed by de-aereating the second homogenous mixture to obtain a de-aerated mixture. This de-aerated mixture was then coated on a PET (polyethylene terephthalate) substrate and dried using hot air to obtain a coated film. Edible film composition was obtained by wrapping Mukhwas in edible film.

### Technical Advancements

The present disclosure described herein above has several technical advantages including, but not limited to, the realization of:—
an edible film composition and a dosage form comprising an edible composition wrapped within an edible composition;
an improved, stable and ready to use edible composition wrapped within an edible film; and
an easy and simple process for the preparation of the edible film composition and the dosage form.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention. The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

We claim:

1. An edible film composition comprising:
a)—at least one cellulose derivative or cellulose based polymer or polysaccharide polymer or starches;
wherein said at least one cellulose derivative or cellulose based polymer or polysaccharide polymer or starches is a film forming substance;
b)—at least one plasticizer; and
c)—water.

2. The edible film composition of claim 1, wherein said edible film disintegrates, or dissolves without leaving substantial residue which needs to be swallowed or ejected from the mouth.

3. The edible film composition of claim 1, wherein said film composition comprises at least one flavouring agent and at least one food colouring agent.

4. The edible film composition of claim 1, wherein said edible film has a thickness ranging from about 1 to about 30 microns.

5. The edible film composition of claim 1, wherein said film composition comprises:
a) at least one cellulose derivative or cellulose based polymer or polysaccharide polymer or starches, in the range of 5 to 30 weight % based on the weight of the edible film composition;
b) at least one plasticizer in the range of 1 to 2 weight % based on the weight of the edible film composition;
c) at least one flavouring agent in the range of 0.01 to 1 weight % based on the weight of the edible film composition;
d) at least one food colouring agent in the range of 0.001 to 0.5 weight % based on the weight of the edible film composition; and
e) water as quantum satis (q.s.).

6. The edible film composition of claim 1, wherein said film forming substance is selected from the group comprising polysaccharide polymer, pullulan, algenates, cellulose polymers, cellulose derivatives like carboxy methyl cellulose and its metal salt derivatives, hydroxy propyl cellulose (HPC), and hydroxy propyl methyl cellulose (EIPMC) or mixture thereof.

7. The edible film composition of claim 1, wherein said plasticizer is selected from the group comprising Triacetin, Glycerlo (GLY), Polyethylene glycol (PEG), and Propylene glycol (PG) or mixture thereof.

8. The edible film composition of claim 1, wherein said flavouring agent is selected from the group comprising natural extrac/flavors, natural identical extracts/flavors and artificial flavors or mixture thereof.

9. The edible film composition of claim 1, wherein said edible film is prepared by the process comprising:
a) adding in a reaction vessel, predetermined amount of cellulose based polymer and water under continuous stirring conditions to obtain a homogeneous mixture;
b) adding predetermined amount of at least one plasticizer, predetermined amount of at least one flavouring agent and predetermined amount of at least one food colouring agent to said homogeneous mixture to obtain a second homogeneous mixture;
c) de-aerating said second homogeneous mixture under vacuum to obtain a de-aerated mixture;
d) coating said de-aerated mixture of step c on a PET (polyethylene terephthalate) substrate and drying using heated air to obtain a coated film;
e) peeling said coated film to obtain said edible film.

10. The edible film composition process as claimed in claim 9, wherein said edible film composition comprises colours and flavours which are shredded into small pieces to make edible glitters or simple flavouring films.
